(19) United States
(12) Patent Application Publication
(10) Pub. No.: US 2022/0298240 A1
(43) Pub. Date: Sep. 22, 2022

(54) Bispecific Chimeric Antigen Receptor

(71) Applicant: Gan & Lee Pharmaceuticals Co., Ltd., Beijing (CN)

(72) Inventors: Jiao Jiao, Beijing (CN); Zhenyang Zhang, Beijing (CN); Zhihao Bao, Beijing (CN)

(21) Appl. No.: 17/596,596

(22) PCT Filed: Jun. 22, 2020
(86) PCT No.: PCT/CN2020/097499

§ 371 Date: Dec. 14, 2021

(30) Foreign Application Priority Data
Jun. 21, 2019 (CN) 201910539645.5

(51) Int. Cl. C07K 16/28; C07K 14/705; C07K 14/725; A61P 35/00; A61P 35/02

(52) U.S. Cl. CPC C07K 16/2803; C07K 14/70517; C07K 14/70578; C07K 14/7051; A61P 35/00; A61P 35/02; A61K 2039/505

(57) ABSTRACT

The present invention relates to a bispecific chimeric antigen receptor, which can target both CD19 and CD22 proteins. T cells expressing the chimeric antigen receptor have good killing effect on tumor cells expressing CD19 and/or CD22 proteins. The chimeric antigen receptor of the present invention can realize a good killing effect on tumor cells with loss of CD19, and provides a more effective therapeutic way for tumor diseases.

### TECHNICAL FIELD

The present invention relates to the field of biomedicine, and in particular to a bispecific chimeric antigen receptor, and an encoding gene, an expression vector, a virus, a cell and a cell population thereof, use thereof in treating diseases and use thereof in preparing a medicament.

### BACKGROUND

T lymphocyte is the natural enemy of tumor cells, and plays a crucial role in immune response to tumors, showing remarkable killing effect on tumor cells. However, when endogenous T cells are used for tumor immunotherapy, the target antigen must be processed to interact with the main histocompatibility complex (MHC) on the surface of the target cell, i.e., “MHC restriction”. However, the process of tumor immunoediting can reduce the expression of MHC on the surface of tumor cells, disrupt the antigen processing and reduce the immunogenicity of the peptide fragments. Over time, this may activate the immune escape mechanism, which allows the tumor cells to escape from the attack of T cells and enable the tumor to rapidly proliferate. Furthermore, the quantity of tumor-specific T cells in the body is small and is further reduced as constantly expressed self-antigens in most tumor cells may lead to neutralization or elimination of T cells targeting these antigens through immune resistance mechanisms. Thus, adoptive T cell immunotherapy, including cytokine-induced killer cells, is not satisfactory for most tumors, although it has demonstrated certain efficacy in the treatment of some tumors.

Chimeric antigen receptor T-cell immunotherapy, or CAR-T, recombines an antigen binding portion for recognizing tumor-associated antigen (TAA, which refers to some tumor cell surface glycoprotein or glycolipid components with slight expression on normal cells but significantly increased expression on tumor cells) with an intracellular signal domain “immunoreceptor tyrosine-based activation motif” (ITAM) in vitro to generate a recombinant plasmid, and then transfects the T cell of a patient with the recombinant plasmid by an in vitro transfection technology, such that the T cells of the patient express a tumor antigen receptor.

After purification and large-scale expansion, the transfected T cells, i.e., CAR-T cells, can specifically recognize tumor-associated antigens, greatly improve the targeting and killing activity and persistence of effector T cells compared with conventional immune cells, and overcome the local tumor immunosuppression microenvironment, thereby breaking host immune resistance state and killing tumor cells.

For CD19-targeting CAR-T, the primary indications are relapsed or refractory acute B-lymphocytic leukemia and relapsed or refractory diffuse large B-cell lymphoma. CD19CAR-T can elevate the long-term survival rate to about 50% for patients with the relapsed or refractory leukemia, and is one of the means with best efficacy at present. However, since the introduction of CD19CAR-T-based immunotherapy, an increasing reduction or loss of CD19 on the surface of tumor cells is observed, eventually leading to relapse. Reduction or loss of CD19 is the primary mechanism of resistance to immunotherapy against CD19. In a recent report where 50 patients showed response to CD19CAR-T therapy, 40% of the patients had relapses in a median follow-up period of 10.6 months, with CD19 protein loss accounting for 65% of the total number of relapses. After relapse, essentially no effect was observed for other therapies.

Therefore, there is an unmet need for treatments against cancers, particularly hematological malignant tumors, and there is a need for drugs with better efficacy.

### SUMMARY

To solve the above problems, in a first aspect, the present invention provides a bispecific chimeric antigen receptor (CAR) comprising an anti-CD22 antigen binding domain, an anti-CD19 antigen binding domain, a hinge region, a transmembrane region, and an intracellular signaling domain. The anti-CD22 antigen binding domain is linked to the anti-CD19 antigen binding domain by a linker sequence selected from one of (GGGS)ₘ, (GGGGS)ₘ, (SSSSG)ₘ, (GSGSA)ₘ and (GGSGG)ₘ, preferably the linker sequence is (GGGGS)ₘ, wherein m is 1 or 2; or the linker sequence between the anti-CD22 antigen binding domain and the anti-CD19 antigen binding domain is selected from any two of (GGGS)ₘ, (GGGGS)ₘ, (SSSSG)ₘ, (GSGSA)ₘ and (GGSGG)ₘ, wherein m is 1.

### Optionally, m in (GGGGS)ₘ is 1.

Optionally, the anti-CD22 antigen binding domain is an anti-CD22 scFv and the anti-CD19 antigen binding domain is an anti-CD19 scFv.
Optionally, the anti-CD22 scFv is a VH-X-VL, wherein X is selected from one or more of (GGGGS)ₙ, (GGGS)ₚ, (SSSSG)_{q}, (GSGSA)ₕ and (GGSGG)ᵢ, preferably, the anti-CD22 scFv is VH-(GGGGS)ₙ-VL; the anti-CD19 scFv is VH-Y-VL, wherein Y is selected from one or more of (GGGGS)ₖ, (GGGS)ᵣ, (SSSSG)ₛ, (GSGSA)ₜ and (GGSGG)ᵥ, preferably, the anti-CD19 scFv is VH-(GGGGS)ₖ-VL, wherein n, p, q, h, i, k, r, s, t and v are each independently an integer not less than 1, preferably n, p, q, h, i, k, r, s, t and v are each independently 2, 3 or 4, and more preferably n, p, q, h, i, k, r, s, t and v are each independently 3.
Optionally, the transmembrane region comprises a human CD8 transmembrane region; preferably, the human CD8 transmembrane region has an amino acid sequence set forth in SEQ ID NO: 8 or SEQ ID NO: 9.

Optionally, the intracellular signaling domain comprises a human 41BB intracellular region (preferably SEQ ID NO: 14); preferably, the intracellular signaling domain further comprises a human CD3ζ intracellular region (preferably SEQ ID NO: 15).

Optionally, the hinge region comprises a human CD8 hinge region; preferably, the human CD8 hinge region has an amino acid sequence set forth in SEQ ID NO: 6 or SEQ ID NO: 7. More preferably, the human CD8 transmembrane region has an amino acid sequence set forth in SEQ ID NO: 9, and the human CD8 hinge region has an amino acid sequence set forth in SEQ ID NO: 7.

Optionally, the amino acid sequence of the chimeric antigen receptor comprises, in the following order: optionally a signal peptide sequence set forth in SEQ ID NO: 1, the amino acid sequence of the anti-CD22 antigen binding domain as described above, the amino acid sequence of the anti-CD19 antigen binding domain as described above, the human CD8 hinge region sequence, the human CD8 transmembrane region sequence, the human 41BB intracellular region sequence and the human CD3ζ intracellular region sequence.

In a second aspect, the present invention provides a polynucleotide sequence comprising a polynucleotide sequence encoding the chimeric antigen receptor according to the first aspect of the present invention.

In a third aspect, the present invention provides a vector comprising the polynucleotide sequence according to the second aspect of the present invention.

In a fourth aspect, the present invention provides a lentivirus or retrovirus comprising the polynucleotide sequence according to the second aspect of the present invention.

In a fifth aspect, the present invention provides a cell comprising the chimeric antigen receptor according to the first aspect of the present invention, the polynucleotide sequence according to the second aspect of the present invention, the vector according to the third aspect of the present invention, or the lentivirus or retrovirus according to the fourth aspect of the present invention.

Optionally, the cell is a T cell.

In a sixth aspect, the present invention provides a cell population comprising at least 1 cell according to the fifth aspect of the present invention.

In a seventh aspect, the present invention provides a pharmaceutical composition comprising the chimeric antigen receptor according to the first aspect of the present invention, the polynucleotide sequence according to the second aspect of the present invention, the vector according to the third aspect of the present invention, the lentivirus or retrovirus according to the fourth aspect of the present invention, the cell according to the fifth aspect of the present invention or the cell population according to the sixth aspect of the present invention, and a pharmaceutically acceptable excipient.

In an eighth aspect, the present invention provides use of the chimeric antigen receptor according to the first aspect of the present invention, the polynucleotide sequence according to the second aspect of the present invention, the vector according to the third aspect of the present invention or the lentivirus or retrovirus according to the fourth aspect of the present invention in preparing the cell according to the fifth aspect of the present invention or the cell population according to the sixth aspect of the present invention.

In a ninth aspect, the present invention provides use of the chimeric antigen receptor according to the first aspect of the present invention, the cell according to the fifth aspect of the invention, the cell population according to the sixth aspect of the present invention or the pharmaceutical composition according to the seventh aspect of the present invention in preparing a medicament for treating a disease mediated by cells expressing CD19 or CD22.

Optionally, the disease mediated by cells expressing CD19 or CD22 is a cancer, preferably the disease is a hematological malignant tumor; more preferably, the disease is B-cell lymphoma, mantle cell lymphoma, acute lymphocytic leukemia, chronic lymphocytic leukemia, hairy cell leukemia, or acute myeloid leukemia; more preferably, the disease is relapsed or refractory acute B-cell lymphocytic leukemia, or relapsed or refractory diffuse large B-cell lymphoma; even more preferably, the disease is a disease with loss of CD19 expression, such as a disease with loss of CD19 expression due to previous treatment.

The inventors found that by ligating the anti-CD19 antigen binding domain and the anti-CD22 antigen binding domain in an order from N terminus to C terminus of the chimeric antigen receptor disclosed herein, the resulting CAR demonstrates an effect comparable to that of a CD19CAR.

The inventors also surprisingly found that: on one hand, the chimeric antigen receptor disclosed herein comprising two antigen binding domains (anti-CD22 antigen binding domain and anti-CD19 antigen binding domain) overcomes the challenge of relapse due to the loss of CD19 antigen caused by use of CD19CAR (hereinafter also referred to as 19CAR) merely comprising the anti-CD19 antigen binding domain; on the other hand, by ligating the anti-CD22 antigen binding domain and the anti-CD19 antigen binding domain in an order from N terminus to C terminus of the chimeric antigen receptor disclosed herein, the present invention can significantly improve the killing efficiency of the obtained bispecific CAR on tumor cells with loss of CD19, as compared to, for example, the approach ligating the anti-CD19 antigen binding domain and the anti-CD22 antigen binding domain in an order from N terminus to C terminus. Furthermore, the inventors also surprisingly found that the killing efficiency of the CAR disclosed herein against tumor cells with loss of CD19 can be further significantly increased by using a linker sequence disclosed herein, such as GGGGS or (GGGGS)₂, between the anti-CD22 antigen binding domain and the anti-CD19 antigen binding domain. In the prior art, since GGGGS and other sequences are generally considered as flexible and protease-resistant linker sequences, researchers would typically use 5 units of GGGGS or other sequences or at least 3 or more units of GGGGS or other sequences as a linker sequence between two antigen binding domains, because it would sufficiently expose the two binding domains of the bispecific CAR, while using a short linker sequence would typically cause the two binding domains to be easily blocked by each other, reducing the binding efficiency of the bispecific CAR to the target antigen. However, the inventors unexpectedly found that using 1 or 2 repeats of GGGGS or other sequences as the linker sequence enhanced the killing efficiency of CAR on tumor cells with loss of CD19, as compared to a linker sequence with 3 or more repeats of GGGGS unit.

The present invention has the following beneficial effects: 1. Compared to the existing CD19CAR, the bispecific CAR disclosed herein can target both CD19 and CD22 antigens, and has higher killing efficiency on tumor cells with loss of CD19 and tumor cells with loss of CD22. 2. Compared to a bispecific CAR obtained by ligating the anti-CD19 antigen binding domain and the anti-CD22 antigen binding domain in an order from N terminus to C terminus (i.e., anti-CD19 antigen binding domain—linker sequence—anti-CD22 antigen binding domain—transmembrane domain—intracellular signal domain), the bispecific CAR disclosed herein has a significantly improved killing effect on tumor cells with loss of CD19. 3. The bispecific CAR has an improved killing effect on tumor cells with loss of CD19 compared to a CAR having 3 or more repeats of GGGGS between the anti-CD22 antigen binding domain and the anti-CD19 antigen binding domain as a linker sequence. 4. When the linker sequence between the VH and VL of each of the anti-CD22 scFv and the anti-CD19 scFv comprises 2-4 units, particularly 3 repeats of GGGGS, the VH and VL of each are better capable of forming an active conformation and thus better binding antigens.

### BRIEF DESCRIPTION OF THE DRAWINGS

FIG. 1: the structural schematic diagrams of CARs in some examples and comparative examples of the present invention, wherein, 1a is an exemplary 19-22CAR, 1b is an exemplary 22-19CAR, 1c is an exemplary 19CAR, and 1d is an exemplary 22CAR.

FIG. 2: the knockout efficiency of CD19 and CD22 genes in CD19⁻CD22⁺ and CD19⁺CD22⁻ lymphoma cells determined by flow cytometry.

FIG. 3: FIG. 3a is the knockout efficiency of CD19 in CD19⁻CD22⁺ Nalm6 human B-lymphocytic leukemia cells determined by flow cytometry, and FIG. 3b is the expression efficiency of CD22 in CD19⁻CD22⁺ Nalm6 human B-lymphocytic leukemia cells determined by flow cytometry.

FIG. 4: the results of an in vivo pharmacodynamic study of R22-19 in killing wild-type Nalm6 human B-lymphocytic leukemia cells.

FIG. 5: the results of an in vivo pharmacodynamic study of R22-19 in killing CD19⁻CD22⁺ Nalm6 human B-lymphocytic leukemia cells.

### DETAILED DESCRIPTION

### Definitions

The term “chimeric antigen receptor” or “CAR” refers to a recombinant polypeptide construct comprising at least an extracellular antigen binding domain, a transmembrane domain, and an intracellular signaling domain.

The term “19-22CAR” refers to a chimeric antigen receptor that comprises, in an order from N terminus to C terminus, anti-CD19 antigen binding domain—anti-CD22 antigen binding domain—transmembrane domain—intracellular signaling domain. The term “19-22CAR-T” refers to a T cell comprising the 19-22CAR.

The term “22-19CAR” refers to a chimeric antigen receptor that comprises, in an order from N terminus to C terminus, anti-CD22 antigen binding domain—anti-CD19 antigen binding domain—transmembrane domain—intracellular signaling domain, wherein 22-19CAR or H22-19CAR indicates that the CD22 antigen is of human origin, and R22-19CAR indicates that the CD22 is of a murine origin. The term “22-19CAR-T” refers to a T cell comprising the 22-19CAR.

The term “CD19CAR” or “19CAR” refers to a chimeric antigen receptor that comprises, in an order from N terminus to C terminus, anti-CD19 antigen binding domain—transmembrane domain—intracellular signaling domain. The term “CD19CAR-T” or “19CAR-T” refers to a T cell comprising a CD19CAR.

The term “CD22CAR” or “22CAR” refers to a chimeric antigen receptor that comprises, in an order from N terminus to C terminus, anti-CD22 antigen binding domain—transmembrane domain—intracellular signaling domain. The term “CD22CAR-T” or “22CAR-T” refers to a T cell comprising a CD22CAR.

The anti-CD22 antigen binding domain and the anti-CD19 antigen binding domain may comprise any antigen binding portion of an anti-CD22 antibody and an anti-CD19 antibody, respectively. The antigen binding portion can be any portion having at least one antigen binding site, such as Fab, F(ab′)₂, dsFv, scFv, diabody and triabody. Preferably, the antigen binding portion is a single-chain variable fragment (scFv). An scFv is a truncated Fab fragment that comprises a heavy chain variable (V) domain and a light chain variable (V) domain of an antibody linked by a synthetic peptide linker (or linker sequence), and can be produced by conventional recombinant DNA techniques. Similarly, a disulfide-stabilized variable fragment (dsFv) can also be prepared by recombinant DNA techniques.

The term “antibody” refers to a protein or polypeptide sequence that is derived from an immunoglobulin molecule and specifically binds to an antigen. Antibodies may be polyclonal or monoclonal, multi-or single-chain, or intact immunoglobulins and may be derived from natural sources or from recombinant sources. The antibody may be a tetramer of immunoglobulin molecules.

The term “antibody heavy chain variable region” or “VH” refers to the larger of the two types of polypeptide chains present in an antibody molecule in its native conformation, which generally determines the class to which the antibody belongs.

The term “antibody light chain variable region” or “VL” refers to the smaller of the two types of polypeptide chains present in an antibody molecule in its native conformation. κ and λ light chains are the two major antibody light chain isotypes.

The term “4-1BB” refers to a member of the Tumor Necrosis Factor Receptor (TNFR) superfamily, which has an amino acid sequence of GenBank Acc. No. AAA62478.2, or an amino acid sequence of a homologous molecule from non-human species such as mouse, rodent, monkey and ape; “4-1BB costimulatory domain” is defined as amino acid residues 214-255 of GenBank Acc. No. AAA62478.2, or an amino acid sequence of a homologous molecule from non-human species such as mouse, rodent, monkey and ape.

As used herein, the term “transmembrane region” refers to a region spanning a cell membrane in a protein sequence, including, but not limited to, the portion of the protein sequence spanning the cell membrane, and 1-20 amino acids on each side of the region. In one embodiment, the transmembrane region is a human CD8 transmembrane region. As used herein, “human CD8 transmembrane region” (or CD8TM) refers to a polypeptide sequence having at least 70%, 80%, 85%, 90%, 95% or 99% homology to a reference sequence (e.g., a portion of a native CD8 protein sequence that spans a cell membrane); preferably, the human CD8 transmembrane region is an amino acid sequence obtained by adding 1-10 amino acid residues at the C terminus of the reference sequence (e.g., a portion of a native CD8 protein sequence that spans a cell membrane).

The term “hinge region” refers to the region between the CH1 and CH2 functional regions of the immunoglobulin heavy chain comprising a large amount of proline. The hinge region is a flexible sequence suitable for binding antigens, and is involved in complement activation. In one embodiment, the hinge region is a human CD8 hinge region, and as used herein, “human CD8 hinge region” (or “CD8Hinge”) refers to a polypeptide sequence having at least 70%, 80%, 85%, 90%, 95% or 99% homology to a reference sequence (e.g., a region of a native CD8 between the CH1 and CH2 functional regions). Preferably, the human CD8 hinge region is an amino acid sequence obtained by adding 1-10 amino acid residues at the N terminus of the reference sequence (e.g., a region of a native CD8 between the CH1 and CH2 functional regions).

The term “CD3ζ” or “CD247” refers to a protein encoded by CD247 gene. In one embodiment, the amino acid sequence of the intracellular domain of human CD3ζ is SEQ ID NO: 15: RVKFSRSADAPAYKQGQNQLYNELNLG RREEYDVLDKRRGRDPEMGGKPRRKNPQEGLYNELQKDKMAEAYSEIGMKGERRRGK GHDGLYQGLSTATKDTYDALHMQALPPR.

The term “expression vector” or “vector” refers to a vector comprising a recombinant polynucleotide comprising an expression control sequence operably linked to a nucleotide sequence to be expressed. Expression vectors contain sufficient cis-regulatory elements for expression, and other elements for expression can be provided by a host cell or in an in vitro expression system. Expression vectors include all expression vectors known in the art to which the recombinant polynucleotide can be incorporated, including cosmid and plasmid (e.g., naked or contained in liposomes).

Both lentiviruses and retroviruses belong to the Retroviridae family. They are typically characterized by the ability that the RNA genomes can be reversely transcribed into cDNA copies that can be stably integrated into the genome of the host cell (generally referred to as stable transfection). Retroviruses are generally classified into two groups: simple retroviruses (sometimes also referred to as oncoviruses or γ-retroviruses, such as murine leukemia virus) and complex retroviruses (such as lentiviruses). The term “lentivirus” refers to species of the Lentiviridae genus. Lentiviruses are unique among retroviruses and are capable of infecting non-dividing cells. They are capable of delivering a considerable amount of genetic information into the DNA of host cells, and are thus one of the most efficient gene delivery vectors. HIV, SIV and FIV are examples of lentiviruses.

The term “linker,” “linker sequence,” or “adapter,” when used along with the description of scFv, refers to a peptide linker consisting of amino acids (such as glycine and/or serine residues) used alone or in combination, to ligate the heavy chain variable region and the light chain variable region. In one embodiment, the linker is a Gly/Ser linker comprising a repeating unit of an amino acid sequence Gly-Gly-Gly-Gly-Ser or GGGGS. As used herein, “GGGGS” refers to the amino acid sequence Gly-Gly-Gly-Gly-Ser, “GGGS” refers to the amino acid sequence Gly-Gly-Gly-Ser, “SSSSG” refers to the amino acid sequence Ser-Ser-Ser-Ser-Gly, “GSGSA” refers to the amino acid sequence Gly-Ser-Gly-Ser-Ala, and “GGSGG” refers to the amino acid sequence Gly-Gly-Ser-Gly-Gly.

The term “effector-to-target ratio” refers to the quantity ratio of effector cells to target cells.

### EXAMPLES

The present invention will be further illustrated by the following examples. It should be noted that the examples are not intended to limit the scope of the present invention.

### Materials and Reagents

The materials and reagents used in the present invention are those which are conventionally available on the market unless otherwise stated. Specific materials and reagents used in the present invention are shown in Table 1.

TABLE 1

| Reagent | Company |
|---|---|
| T cells | Guangzhou Leide Biological |
| | Technology Co., Ltd. |
| Anti-CD3/CD28 magnetic | Novoprotein Scientific (Shanghai) |
| bead | Inc. |
| Romas lymphoma cells | American Type Culture Collection |
| | (ATCC) |
| Plasmid pMD2G, plasmid | BioFeng Technology Co., Ltd. |
| psPAX2 | |
| Plasmid pCDH-CMV-MCS | System Bioscience (SBI), LLC. |
| Plasmid pCDH-EF1α | System Bioscience (SBI), LLC. |
| CAS9 protein | Nanjing Genscript Biotechnology Co., Ltd. |
| SgRNA IVT kit | Nanjing Genscript Biotechnology Co., Ltd. |
| crRNA | Nanjing Genscript Biotechnology Co., Ltd. |
| tracrRNA | Nanjing Genscript Biotechnology Co., Ltd. |
| APC-anti-CD3 | STEMCELL |
| APC-anti-CD22 | STEMCELL |
| PE-anti-CD3 | STEMCELL |
| FITC-anti-HLA1 | STEMCELL |
| PE-anti-b2m | STEMCELL |
| PE-anti-CD19 | STEMCELL |
| FITC-anti-TCR | STEMCELL |

### Example 1. Preparation of 22-19CAR-T cells

### Preparation of Vector

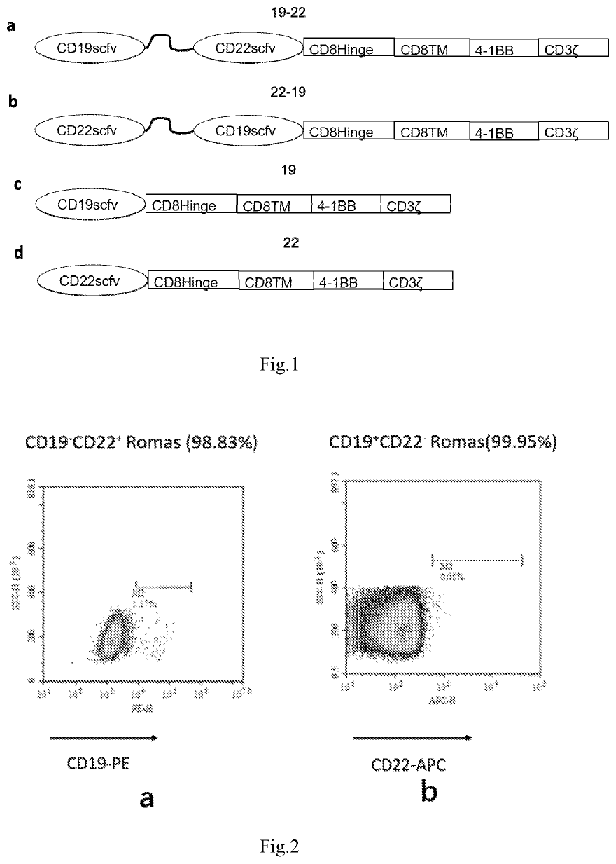

For the bispecific CAR with an order of domains shown in FIG. 1B and a specific sequence set forth in SEQ ID NO: 10 (wherein the VH sequence of CD22 scFv is set forth in SEQ ID NO: 2, the VL sequence of CD22 scFv is set forth in SEQ ID NO: 3, the VH sequence of CD19 scFv is set forth in SEQ ID NO: 5, the VL sequence of CD19 scFv is set forth in SEQ ID NO: 4, the sequence of CD8hinge is set forth in SEQ ID NO: 7, the sequence of CD8TM is set forth in SEQ ID NO: 9, the sequence of 4-1BB is set forth in SEQ ID NO: 14, the sequence of CD3ζ is set forth in SEQ ID NO: 15, and the linker sequence between the anti-CD22 domain and the anti-CD19 domain is GGGGS), a 22-19CAR gene was synthesized. The obtained gene was then ligated to a lentiviral vector pCDH-CMV-MCS. The CAR gene was subcloned into the MCS (multiple cloning site) of the pCDH-CMV-MCS lentiviral expression vector by digestion (XbaI and EcoRI)-ligation. The cells were transformed and monoclones were selected. The plasmids were extracted and sequenced. Plasmids with correct sequences were selected and the corresponding strains were preserved and cultured. Plasmids were extracted from the cultures for lentiviral packaging.

### Lentiviral Packaging

Lentiviral packaging was performed according to the method disclosed in Yang S, Shi H, Chu X, et al., A rapid and efficient polyethylenimine-based transfection method to prepare lentiviral or retroviral vectors: useful for making iPS cells and transduction of primary cells, [J]. Biotechnology Letters, 2016, 38(9): 1631-1641.

### Infection of T Cells

The harvested virus supernatant was added to an ultracentrifuge tube, centrifuged at 25000 rpm at 4° C. for 2 h, the supernatant was discarded, and the residual was dissolved in sterile PBS (phosphate-buffered saline). Then, 1×10⁵ T cells were mixed with 300-400 μL of virus concentrate and cultured overnight in an incubator. Infected cells were harvested, washed and resuspended in PBS. 1 μg/mL CD19-Fc fusion protein (purchased from NovoProtein) was added, and the mixture was mixed and incubated at 4° C. for 1 h. The cells were washed and resuspended in PBS. The supernatant was discarded, and the cells were resuspended in 100 μL of PBS. 1 μg of PE-labeled anti-human IgG-Fc antibody for flow cytometry (purchased from Invitrogen) was added, and the cells were incubated at 4° C. away from the light for 15 min, washed with PBS and resuspended in PBS again. Selection of infected cells: Infected cells were harvested, washed and resuspended in PBS. 1 μg/mL CD19-Fc fusion protein (purchased from NovoProtein) was added, and the mixture was mixed and incubated at 4° C. for 1 h. The cells were washed and resuspended in PBS. The supernatant was discarded, and the cells were resuspended in 100 μL of PBS. 1 μg of PE-labeled anti-human IgG-Fc antibody for flow cytometry was added, and the cells were incubated at 4° C. away from the light for 15 min, washed with PBS and resuspended in PBS again. PE-positive cells (i.e., CAR-T cells) were selected by EasySep™ Human PE Positive Selection Kit II (STEMCELL).

### Example 2. Preparation of 22-19CAR2-T cells

22-19CAR2-T cells was prepared following a procedure similar to Example 1. The CAR expressed by the 22-19CAR2-T cells comprised domains ligated in the order shown in FIG. 1B, differing from the sequence of the CAR in Example 1 in that the linker sequence (or linker) between the anti-CD22 domain and the anti-CD19 domain was (GGGGS)₂.

### Comparative Example 1. Preparation of 19-22CAR-T Cells

Bispecific 19-22CAR-T cells were prepared following a procedure similar to Example 1, and the CAR expressed by the bispecific 19-22CAR-T cells comprised domains ligated in the order shown in FIG. 1a. The sequences of the domains and the linker were the same as those in Example 1.

### Comparative Example 2. Preparation of CD22CAR-T Cells

CD22CAR-T cells were prepared following a procedure similar to Example 1, and the CAR expressed by the CD22CAR-T cells comprised domains ligated in the order shown in FIG. 1d. The sequences of the domains and the linker were the same as those in Example 1.

### Comparative Example 3. Preparation of CD19CAR-T Cells

CD19CAR-T cells were prepared following a procedure similar to Example 1, and the CAR expressed by the CD19CAR-T cells comprised domains ligated in the order shown in FIG. 1c. The sequences of the domains and the linker were the same as those in Example 1.

### Comparative Example 4. Preparation of 22-19CAR3-T Cells

22-19CAR3-T cells was prepared following a procedure similar to Example 1. The CAR expressed by the 22-19CAR3-T cells comprised domains ligated in the order shown in FIG. 1B, differing from the sequence of the CAR in Example 1 in that the linker sequence (or linker) between the anti-CD22 domain and the anti-CD19 domain was (GGGGS)₃.

### Comparative Example 5. Preparation of 22-19CAR4-T Cells

22-19CAR4-T cells was prepared following a procedure similar to Example 1. The CAR expressed by the 22-19CAR4-T cells comprised domains ligated in the order shown in FIG. 1B, differing from the sequence of the CAR in Example 1 in that the linker sequence (or linker) between the anti-CD22 domain and the anti-CD19 domain was (GGGGS)₄.

### Comparative Example 6. Preparation of 22-19CAR5-T Cells

22-19CAR5-T cells was prepared following a procedure similar to Example 1. The CAR expressed by the 22-19CAR5-T cells comprised domains ligated in the order shown in FIG. 1b, differing from the sequence of the CAR in Example 1 in that the linker sequence (or linker) between the anti-CD22 domain and the anti-CD19 domain was (GGGGS)₅.

### Example 3. In Vitro Killing Activity of CAR-T Cells

1. Construction of CD19⁻CD22⁺ and CD19⁺CD22⁻ Cell Lines

Using Romas lymphoma cells (wild-type Romas lymphoma cells are CD19⁺ and CD22⁺; in the present invention, CD19⁺ Romas lymphoma cell also sometimes denotes a naive Romas lymphoma cell without gene knockout), CD19 and CD22 genes were knocked out according to the method disclosed in Liu X, Zhang Y, Cheng C, et al., CRISPR-Cas9-mediated multiplex gene editing in CAR-T cells, [J]. Cell Research, 2017, 27(1): 154, so as to obtain CD19⁻CD22⁺ and CD19⁺CD22⁻ lymphoma cells. After PE-anti-CD19 or APC-anti-CD22 antibody staining, the knockout efficiency of CD19 and CD22 genes was measured by flow cytometry (see FIG. 2). After sorting by flow cytometry, the CD19 knockout efficiency was 98.83%, and the CD22 knockout efficiency was 99.95%. The knockout efficiency measured by flow cytometry sorting was greater than 98%.

### 2. In Vitro Killing Assay of CAR-T Cells

22-19CAR-T cells, 19-22CAR-T cells, CD22CAR-T cells and CD19CAR-T cells prepared in Example 1 and Comparative Examples 1-3 and untransfected T cells were used as effector cells, and Romas lymphoma cells, CD19⁻CD22⁺ lymphoma cells, and CD19⁺CD22⁻ lymphoma cells were used as target cells. Effector cells and target cells were co-incubated in a 96-well plate in different ratios (effector-to-target ratios) for 48 h, and stained with PE-anti-CD19 or APC-anti-CD22 antibody. Apoptosis and necrosis of the target cells and the effector cells were determined by fluorescence-activated cell sorting (FACS) after staining.

### 1). Killing Effect Comparison of 22-19CAR-T Cells and Untransfected T Cells

After 22-19CAR-T cells and untransfected T cells were co-incubated with CD19⁺CD22⁻ lymphoma cells or CD19⁻CD22⁺ lymphoma cells in a ratio of 1:1, 5:1 or 10:1 in 96-well plates for 48 h, the killing effect of 22-19CAR-T cells on lymphoma cells was determined by flow cytometry, giving the following results: at effector-to-target ratios of 1:1, 5:1 and 10:1, the 22-19CAR-T cells were capable of killing the vast majority of CD19⁺CD22⁻ lymphoma cells and CD19⁻CD22⁺ lymphoma cells as compared to the untransfected T cell group, as shown in Tables 2 and 3.

TABLE 2
Killing effect of 22-19CAR-T cells and untransfected
T cells on CD19⁻CD22⁺ lymphoma cells

| Effector-to-target ratio | 1:1 | 5:1 | 10:1 |
|---|---|---|---|
| 22-19CAR-T/ | 99.2% | 99.5% | 99.4% |
| CD19⁻CD22⁺ lymphoma cells | | | |
| Untransfected T cells/ | 0 | 0 | 0 |
| CD19⁻CD22⁺ lymphoma cells | | | |

TABLE 3
Killing effect of 22-19CAR-T cells and untransfected
T cells on CD19⁺CD22⁻ lymphoma cells

| Effector-to-target ratio | 1:1 | 5:1 | 10:1 |
|---|---|---|---|
| 22-19CAR-T/ | 99.5% | 99.6% | 99.3% |
| CD19⁺CD22⁻ lymphoma cell | | | |
| Untransfected T cells/ | 0 | 0 | 0 |
| CD19⁺CD22⁻ lymphoma cell | | | |

### 2) Killing Effect Comparison of 22-19CAR-T and CD19CAR-T

After 22-19CAR-T and CD19CAR-T were co-incubated with CD19⁺ Romas lymphoma cells (i.e., naive Romas lymphoma cells without gene knockout) in a ratio of 1:2, 1:1 or 3:1 in 96-well plates for 48 h, the cells were harvested, washed with PBS and resuspended, incubated with PE-anti-CD19 antibody for flow cytometry at 4° C. for 30 min. The killing effect of 22-19CAR-T and CD19CAR-T on CD19⁺ Romas lymphoma cells was determined by flow cytometry, giving the following results: at effector-to-target ratios of 1:2, 1:1 and 3:1, 22-19CAR-T and CD19CAR-T demonstrated similar effects, and 22-19CAR-T were significantly capable of killing CD19⁺ Romas lymphoma cells, as shown in Table 4.

TABLE 4
Killing effect of 22-19CAR-T and CD19CAR-T
on CD19⁺ Ramos lymphoma cells

| Effector-to-target ratio | 1:1 | 2:1 | 3:1 |
|---|---|---|---|
| 22-19CAR-T/ | 99.3% | 99.6% | 99.4% |
| CD19⁺ Romas lymphoma cells | | | |
| CD19CAR-T/ | 99.5% | 99.3% | 99.8% |
| CD19⁺ Romas lymphoma cells | | | |

### 3) Killing Effect Comparison of 22-19CAR-T Cells to 19-22CAR-T Cells, CD19CAR-T Cells and CD22CAR-T Cells

After 22-19CAR-T cells, 19-22CAR-T cells, CD19CAR-T cells, CD22CAR-T cells and untransfected T cells were co-incubated with CD19⁺CD22⁻ lymphoma cells or CD19⁻CD22⁺ lymphoma cells in an effector-to-target ratio of 5:1 in 96-well plates for 48 h, the cells were harvested, washed with PBS and resuspended, and the killing effect of CAR-T on CD19⁺CD22⁻ lymphoma cells was determined by flow cytometry, giving the result that: when comparing 22-19CAR-T cells with 19-22CAR-T cells, CD19CAR-T cells, CD22CAR-T cells and blank T cells, the 19-22CAR-T cells, CD19CAR-T cells and blank T cells were not capable of effectively killing CD19⁻CD22⁺ lymphoma cells; CD22CART was capable of killing CD19⁻CD22⁺ lymphoma cells, but was not capable of killing CD19⁺CD22⁻ lymphoma cells; 22-19CAR-T cells were capable of killing both CD19⁺CD22⁻ and CD19⁻CD22⁺ lymphoma cells. (Table 5)

TABLE 5
Killing effect of 22-19CAR-T cells, 19-22CAR-T cells and untransfected
T cells on CD19⁺CD22⁻ lymphoma cells and CD19⁻CD22⁺ lymphoma cells

| | Killing efficiency | | | | | |
|---|---|---|---|---|---|---|
| | | | | | Untransfected | Untransfected |
| | 22-19CAR-T + | 22-19CAR-T + | 19-22CAR-T + | 19-22CAR-T + | T cells + | T cells + |
| | CD19⁺CD22⁻ | CD19⁻CD22⁺ | CD19⁺CD22⁻ | CD19⁻CD22⁺ | CD19⁺CD22⁻ | CD19⁻CD22⁺ |
| Cell line | lymphoma cell | lymphoma cell | lymphoma cell | lymphoma cell | lymphoma cell | lymphoma cell |
| Effector- | 99.6% | 99.5% | 99.3% | 0 | 0 | 0 |
| to-target | | | | | | |
| ratio of | | | | | | |
| 5:1 | | | | | | |

### 4) Killing Effect Comparison of Examples 1-2 and Comparative Examples 4-6

The killing effect of Examples 1-2 and Comparative Examples 4-6 on CD19⁺CD22⁻ lymphoma cells and CD19⁻CD22⁺ lymphoma cells was determined at an effector-to-target ratio of 5:1 using a procedure similar to that in the killing effect comparison experiment in section 3) above. The results are shown in Table 6.

TABLE 6
Effect of different linker sequences between anti-CD22 and anti-CD19 domains
on CAR-T killing efficiency

| | Linker sequence | | |
|---|---|---|---|
| | between anti-CD22 | Killing efficiency | Killing efficiency |
| Examples or | and anti-CD19 | on CD19⁺CD22⁻ | on CD19⁻CD22⁺ |
| comparative examples | domains | lymphoma cells | lymphoma cells |
| Example 1 | (GGGGS)₁ | 99.6% | 99.5% |
| Example 2 | (GGGGS)₂ | 98.7% | 81.8% |
| Comparative example 4 | (GGGGS)₃ | 96.9% | 68.1% |
| Comparative example 5 | (GGGGS)₄ | 98.2% | 52.0% |
| Comparative example 6 | (GGGGS)₅ | 97.5% | 27.0% |

As can be seen from Table 6 above, the CARs with a linker sequence of 1 or 2 units of (GGGGS) between the anti-CD22 domain and the anti-CD19 domain of Examples 1-2 had a comparable effect in killing CD19⁺CD22⁻ lymphoma cells but a significantly improved effect in killing CD19⁻CD22⁺ lymphoma cells as compared to the CARs with a linker sequence of 3-5 units of (GGGGS) in comparative Examples 4-6. It can be expected that the CAR-T cells of Examples 1-2 of the present invention will have a better therapeutic effect in patients with relapse due to loss of CD19 protein.

### Example 4. Preparation of H22-19CAR-T Cells

H22-19CAR-T cells were prepared by repeating the procedures in Example 1 except for using the CAR in Example 1 and a lentiviral vector pCDH-EF1α (in which the CAR gene was subcloned into the MCS (multiple cloning site) of lentiviral expression vector pCDH-EF1α) by digestion-ligation.

### Example 5. Preparation of R22-19CAR-T Cells

R22-19CAR-T cells were prepared by repeating the procedures in Example 1 except for using a bispecific CAR with the specific sequence of SEQ ID NO: 13 (in which the VH sequence of the murine anti-CD22 binding domain is set forth in SEQ ID NO: 12, the VL sequence is set forth in SEQ ID NO: 11; the VH sequence of the anti-CD19 binding domain is set forth in SEQ ID NO: 5; and the VL sequence is set forth in SEQ ID NO: 4) and a lentiviral vector pCDH-EF1α (in which the CAR gene was subcloned into the MCS (multiple cloning site) of lentiviral expression vector pCDH-EF1α) by digestion-ligation.

### Example 6. In Vitro Killing Activity of CAR-T Cells

1. Construction of CD19⁻CD22⁺ Nalm6 Human B-Lymphocytic Leukemia Cell Line and CD19⁻CD22⁺ Romas Lymphoma Cell Line

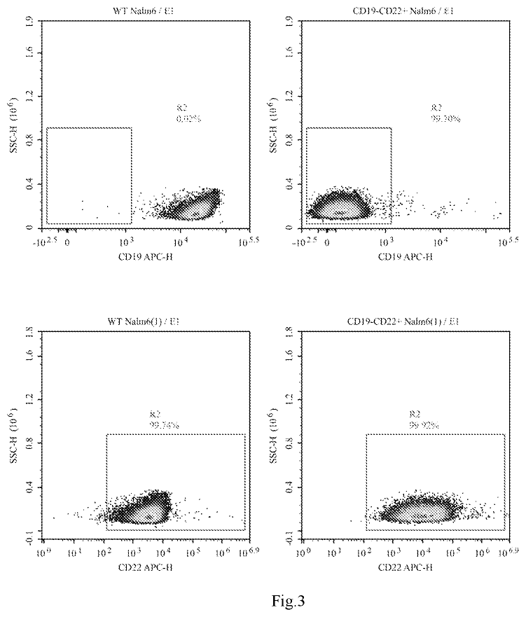

Using Nalm6 human B-lymphocytic leukemia cells (ATCC), the CD19 gene was knocked out according to the method disclosed in Liu X, Zhang Y, Cheng C, et al., CRISPR-Cas9-mediated multiplex gene editing in CAR-T cells, [J]. Cell Research, 2017, 27(1): 154, so as to obtain the CD19⁻CD22⁺ Nalm6 human B-lymphocytic leukemia cells. After PE-anti-CD19 and APC-anti-CD22 antibody staining, CD19 gene knockout efficiency and CD22 gene expression efficiency were measured by flow cytometry (see FIG. 3). The CD19 knockout efficiency was 99.7%, and the CD22 expression efficiency was 99.92%. The CD19 knockout efficiency measured by flow cytometry sorting was greater than 99%.

CD19⁻CD22⁺ Romas lymphoma cells were constructed as in Example 3.

### 3. In Vitro Killing Assay of CAR-T Cells

H22-19CAR-T cells, R22-19CAR-T cells and CD19CAR-T cells prepared in Examples 4-5 and Comparative Example 3 and untransfected T cells were used as effector cells, and CD19⁺ Nalm6 human B-lymphocytic leukemia cells (i.e., wild-type Nalm6 human B-lymphocytic leukemia cells) and CD19⁻CD22⁺ Nalm6 human B-lymphocytic leukemia cells were used as target cells. Effector cells and target cells were co-incubated in a 96-well plate in an effector-to-target ratio of 1:1 for 48 h, and stained with PE-anti-CD19 or APC-anti-CD22 antibody. Apoptosis and necrosis of the target cells and the effector cells were determined by fluorescence-activated cell sorting (FACS) after staining.

1) Killing Effect Comparison of H22-19CAR-T, R22-19CAR-T, CD19CAR-T and Untransfected T cells on CD19⁺ Nalm6 Human B-Lymphocytic Leukemia Cells

After H22-19CAR-T cells, R22-19CAR-T cells, CD19CAR-T cells and untransfected T cells were co-incubated with CD19⁺Nalm6 human B-lymphocytic leukemia cells (i.e., naive Nalm6 human B-lymphocytic leukemia cells without gene knockout) in a ratio of 1:1 in 96 well plates for 48 h, PE-anti-CD19 antibody for flow cytometry was added and the mixture was incubated at 4° C. for 30 min. The killing effect of H22-19CAR-T cells, R22-19CAR-T cells, CD19CAR-T cells and untransfected T cells on CD19⁺ Nalm6 human B-lymphocytic leukemia cells was determined by flow cytometry, giving the following results: in an effector-to-target ratio of 1:1, H22-19CAR-T cells, R22-19CAR-T cells, CD19CAR-T cells were capable of killing the vast majority of CD19+ Nalm6 human B-lymphocytic leukemia cells as compared to the untransfected T cell group, as shown in Table 7.

TABLE 7
Killing effect of H22-19CAR-T cells, R22-19CAR-T cells, CD19CAR-T cells, and
untransfected T cells on CD19⁺ Nalm6 human B-lymphocytic leukemia cells

| | Killing efficiency | | | |
|---|---|---|---|---|
| | | | | Untransfected |
| | H22-19CAR-T + | R22-19CAR-T + | CD19CAR-T + | T cells + |
| | CD19⁺ | CD19⁺ | CD19⁺ | CD19⁺ |
| | Nalm6 human | Nalm6 human | Nalm6 human | Nalm6 human |
| | B-lymphocytic | B-lymphocytic | B-lymphocytic | B-lymphocytic |
| Cell line | leukemia cells | leukemia cells | leukemia cells | leukemia cells |
| Effector-to-target | 99.9% | 99.7% | 99.9% | 0 |
| ratio of 1:1 | | | | |

2) Killing Effect Comparison of H22-19CAR-T Cells, R22-19CAR-T Cells, CD19CAR-T Cells and Untransfected T Cells on CD19⁻CD22⁺ Nalm6 Human B-Lymphocytic Leukemia Cells

After H22-19CAR-T cells, R22-19CAR-T cells, CD19CAR-T cells and untransfected T cells were co-incubated with CD19⁻CD22⁺ Nalm6 human B-lymphocytic leukemia cells in a ratio of 1:1 in 96 well plates for 48 h, APC-anti-CD22 antibody for flow cytometry was added and the mixture was incubated at 4° C. for 30 min. The killing effect of H22-19CAR-T cells, R22-19CAR-T cells, 19CAR-T cells and untransfected T cells on CD19⁻CD22⁺ Nalm6 human B-lymphocytic leukemia cells was determined by flow cytometry, giving the following results: in an effector-to-target ratio of 1:1, as compared to the untransfected T cell group, H22-19CAR-T cells and R22-19CAR-T cells were capable of killing the vast majority of CD19⁻CD22⁺ Nalm6 human B-lymphocytic leukemia cells, but CD19CAR-T cells were not capable of killing CD19⁻CD22⁺ Nalm6 human B-lymphocytic leukemia cells, as shown in Table 8.

TABLE 8
Killing effect of H22-19CAR-T, R22-19CAR-T, CD19CAR-T and untransfected T
cells on CD19⁻CD22⁺ Nalm6 human B-lymphocytic leukemia cells

| | Killing efficiency | | | |
|---|---|---|---|---|
| | | | | Untransfected |
| | H22-19CAR-T + | R22-19CAR-T + | 19CAR-T + | T cells + |
| | CD19⁻CD22⁺ | CD19⁻CD22⁺ | CD19⁻CD22⁺ | CD19⁻CD22⁺ |
| | Nalm6 human | Nalm6 human | Nalm6 human | Nalm6 human |
| | B-lymphocytic | B-lymphocytic | B-lymphocytic | B-lymphocytic |
| Cell line | leukemia cells | leukemia cells | leukemia cells | leukemia cells |
| Effector-to-target | 91.9% | 95.14% | 0 | 0 |
| ratio of 1:1 | | | | |

### 3) Killing Effect Comparison of H22-19CAR-T, R22-19CAR-T, CD19CAR-T and Untransfected T Cells on CD19⁺ Romas Lymphoma Cells

After H22-19CAR-T, R22-19CAR-T, CD19CAR-T, CD22CAR-T and untransfected T cells were co-incubated with CD19⁺ Romas lymphoma cells (i.e., naive lymphoma cells without gene knockout) in an effector-to-target ratio of 1:1 in 96-well plates for 48 h, the cells were harvested, washed with PBS and resuspended. The PE-anti-CD19 antibody for flow cytometry was added and the mixture was incubated at 4° C. for 30 min. The killing effect of CAR-T on CD19⁺Romas lymphoma cells was determined by flow cytometry, giving the following results: when comparing H22-19CAR-T, R22-19CAR-T and CD19CAR-T with untransfected T cells, H22-19CAR-T, R22-19CAR-T and CD19CAR-T were all capable of efficiently killing CD19⁺ Romas lymphoma cells (Table 9).

TABLE 9
Killing effect of H22-19CAR-T, R22-19CAR-T, CD19CAR-T
and untransfected T cells on CD19⁺ Romas lymphoma cells

| | Killing efficiency | | | |
|---|---|---|---|---|
| | | | | Untransfected |
| | H22-19CAR-T + | R22-19CAR-T + | CD19CAR-T + | T cells + |
| | CD19⁺ Romas | CD19⁺ Romas | CD19⁺ Romas | CD19⁺ Romas |
| Cell line | lymphoma cells | lymphoma cells | lymphoma cells | lymphoma cells |
| Effector-to-target | 94.9% | 91.87% | 94.8% | 0 |
| ratio of 1:1 | | | | |

4) Killing Effect Comparison of H22-19CAR-T, R22-19CAR-T, CD19CAR-T and Untransfected T Cells on CD19⁻CD22⁺ Romas Lymphoma Cells

After H22-19CAR-T cells, R22-19CAR-T cells, CD19CAR-T cells and untransfected T cells were co-incubated with CD19⁻CD22⁺ Romas lymphoma cells in a ratio of 1:1 in 96 well plates for 48 h, APC-anti-CD22 antibody for flow cytometry was added and the mixture was incubated at 4° C. for 30 min. The killing effect of H22-19CAR-T cells, R22-19CAR-T cells, CD19CAR-T cells and untransfected T cells on CD19⁻CD22⁺ Romas lymphoma cells was determined by flow cytometry, giving the following results: at an effector-to-target ratio of 1:1, H22-19CAR-T cells and R22-19CAR-T cells were capable of killing the vast majority of CD19⁻CD22⁺ Romas lymphoma cells as compared to untransfected T cells, but 19CAR-T cells were comparable to untransfected T cells and were not capable of killing CD19⁻CD22+ Romas lymphoma cells, as shown in Table 10.

TABLE 10
Killing effect of H22-19CAR-T, R22-19CAR-T, CD19CAR-T and untransfected
T cells on CD19⁻CD22⁺ Romas lymphoma cells

| | Killing efficiency | | | |
|---|---|---|---|---|
| | | | | Untransfected |
| | H22-19CAR-T + | R22-19CAR-T + | 19CAR-T + | T cells + |
| | CD19⁻CD22⁺ Romas | CD19⁻CD22⁺ Romas | CD19⁻CD22⁺ Romas | CD19⁻CD22⁺ Romas |
| Cell line | lymphoma cells | lymphoma cells | lymphoma cells | lymphoma cells |
| Effector-to-target | 75.9% | 82.06% | 0 | 0 |
| ratio of 1:1 | | | | |

As can be seen from Tables 7 and 8 above, the CAR-T cells prepared with the anti-CD22-CD19 structures in Examples 4-5, relative to the CD19CAR-T cells in Comparative Example 3, had comparable efficiency in killing CD19⁺ Nalm6 human B-lymphocytic leukemia cells, but significantly improved efficiency in killing CD19⁻CD22⁺ Nalm6 lymphoma cells. As can be seen from Tables 9 and 10, the CAR-T cells prepared in Examples 4-5 and the CD19CAR-T cells in Comparative Example 3 are all capable of killing the vast majority of CD19⁺ Romas lymphoma cells, but the CD19CAR-T cells in Comparative Example 3 were not capable of killing CD19⁻CD22⁺ Romas lymphoma cells, and only the CAR-T cells prepared with the anti-CD22-CD19 structure in Examples 4-5 had a killing function on CD19⁻CD22⁺ Romas lymphoma cells. It can be expected that the CAR-T cells in Examples 4-5 of the present invention will have a superior therapeutic effect on patients with relapse due to loss of CD19 protein.

### Example 7. In Vivo Pharmacodynamic Study of R22-19 in Killing Wild-Type Nalm6 Human B-Lymphocytic Leukemia Cells AND CD19⁻CD22⁺ Nalm6 Human B-Lymphocytic Leukemia Cells

### 1. In Vivo Pharmacodynamic Study of R22-19 in Killing Wild-Type Nalm6

15 NSG mice (from Biocytogen) aged 6 weeks were injected with 5×10⁵ wild-type Nalm6 human B-lymphocytic leukemia cells per NSG mouse through tail vein. After three days, the 15 NSG mice were randomized into three groups of 5. The three groups were injected with untransfected T cells, CD19CAR-T cells and R22-19CAR-T cells, respectively, each mouse receiving 1×10⁷ CAR-T cells through tail vein. The death status was observed and recorded. The results of the in vivo pharmacodynamic study of R22-19 in killing wild-type Nalm6 are shown in FIG. 4. Results: The first death occurred on Day 29 after receiving Nalm6 human B-lymphocytic leukemia cells through tail vein for the T group, on Day 42 for the R22-19CAR-T group, and on Day 43 for the CD19CAR-T group, respectively. In conclusion, the overall survival of mice bearing wild-type Nalm6 human B-lymphocytic leukemia cells in the R22-19CAR-T group was similar to that of the CD19CAR-T group, and the overall survival of mice in the R22-19CAR-T group was superior to that of the T group, indicating that R22-19CAR-T was capable of effectively killing wild-type Nalm6 cancer cells in mice, significantly prolonging the survival of mice bearing wild-type Nalm6 human B-lymphocytic leukemia cells.

2) In Vivo Pharmacodynamic Study of R22-19 in Killing CD19⁻CD22⁺ Nalm6 Human B-Lymphocytic Leukemia Cells

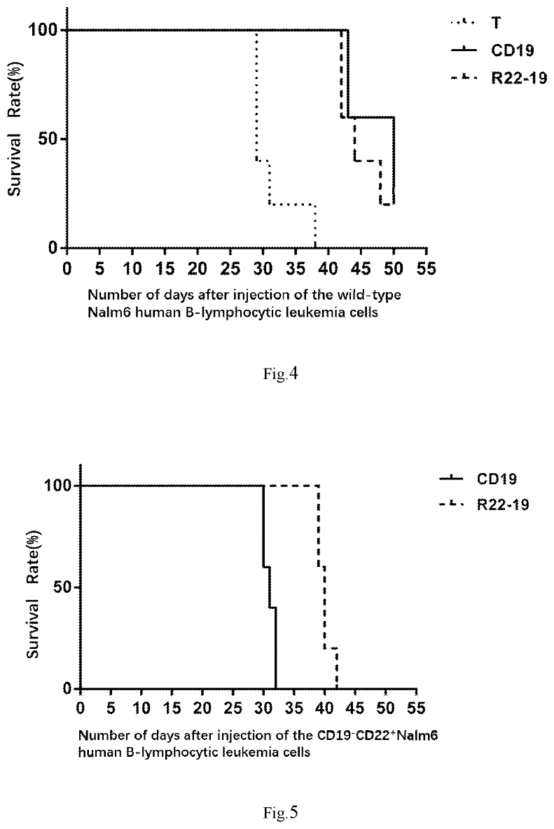

10 NSG mice (from the Biocytogen) aged 6 weeks were injected with 5×10⁵ CD19⁻CD22⁺ Nalm6 human B-lymphocytic leukemia cells per NSG mouse through tail vein. After three days, the 10 NSG mice were randomized into two groups of 5. The two groups were injected with CD19CAR-T cells and R22-19CAR-T cells, respectively, each mouse receiving 1×10⁷ CAR-T cells through tail vein. The death status was observed and recorded. The results of the in vivo pharmacodynamic study of R22-19CAR-T cells in killing CD19⁻CD22⁺ Nalm6 are shown in FIG. 5. Results: All the mice in the CD19CAR-T group died on Day 32 after receiving CD19⁻CD22⁺ Nalm6 cancer cells, while the first death in the R22-19CAR-T group occurred on Day 39 after receiving CD19⁻CD22⁺ Nalm6 cancer cells. In conclusion, the overall survival of mice in the R22-19CAR-T group was superior to that in the CD19CAR-T group, indicating that R22-19CAR-T was capable of effectively killing Nalm6 cancer cells with loss of CD19 protein but expression of CD22 protein in mice, significantly prolonging the survival of mice bearing CD19⁻CD22⁺ Nalm6 cancer cells.

The flow cytometric procedures for determining killing activity are as follows:

The CAR-T cells and cancer cells were co-incubated in 96-well plates in certain effector-to-target ratios, and the cancer cells were incubated in another 96-well plate alone. After 48 h, the cells were washed twice by using FACS buffer and incubated with corresponding antibodies for flow cytometry for 30 min at 4° C. The number of the cancer cells was detected by flow cytometry after the cells were washed twice using the FACS buffer. The ability of the CAR-T cell to kill cancer cells in vitro was evaluated according to a formula calculating the CAR-T cell killing rate.

CAR-T cell killing rate=(number of cancer cells incubated alone−number of cancer cells in co-incubation group)/number of cancer cells incubated alone

The present invention has been illustrated by the above examples, but it should be understood that the above examples are for illustrative and descriptive purposes only and are not intended to limit the present invention to the scope of the described examples. Furthermore, it will be understood by those skilled in the art that the present invention is not limited to the examples described above, and that many variations and modifications can be made in accordance with the teachings of the present invention, all of which fall within the scope of the present invention as claimed. The protection scope of the present invention is defined by the appended claims and equivalents thereof.

1. A bispecific chimeric antigen receptor, comprising, in an order from N terminus to C terminus: an anti-CD22 antigen binding domain, a linker sequence, an anti-CD19 antigen binding domain, a hinge region, a transmembrane region, and an intracellular signaling domain, wherein the linker sequence between the anti-CD22 antigen binding domain and the anti-CD19 antigen binding domain comprises one selected from (GGGS)ₘ, (GGGGS)ₘ, (SSSSG)ₘ, (GSGSA)ₘ and (GGSGG)ₘ, and m is 1 or 2; or
the linker sequence between the anti-CD22 antigen binding domain and the anti-CD19 antigen binding domain comprises two selected from (GGGS)ₘ, (GGGGS)ₘ, (SSSSG)ₘ, (GSGSA)ₘ and (GGSGG)ₘ, and m is 1;
alternatively, the anti-CD22 antigen binding domain and the anti-CD19 antigen binding domain are interchanged in position.

2. The chimeric antigen receptor according to claim 1, wherein the anti-CD22 antigen binding domain is an anti-CD22 say and the anti-CD19 antigen binding domain is an anti-CD19 scFv.

3. The chimeric antigen receptor according to claim 2, wherein the anti-CD22 scFv comprises VH-X-VL, wherein X comprises one or more of (GGGGS)ₙ, (GGGS)ₚ, (SSSSG)_{q}, (GSGSA)ₕ and (GGSGG)ᵢ and the anti-CD19 scFv comprises VH-Y-VL, wherein Y comprises one or more of (GGGGS)ₖ, (GGGS)ₜ, (SSSSG)ₛ, (GSGSA)ₜ and (GGSGG)ᵥ, wherein n, p, q, h, k, r, s, t and v are each independently an integer not less than 1.

4. The chimeric antigen receptor according to claim 1, wherein the heavy chain variable region (VH) of the anti-CD22 antigen binding domain comprises the amino acid sequence of SEQ ID NO: 2; and/or the light chain variable region (VL) of the anti-CD22 antigen binding domain comprises the amino acid sequence of SEQ ID NO: 3; or the heavy chain variable region (VH) of the anti-CD22 antigen binding domain comprises the amino acid sequence of SEQ ID NO: 12; and/or the light chain variable region (VL) of the anti-CD22 antigen binding domain comprises the amino acid sequence of SEQ ID NO: 11.

5. The chimeric antigen receptor according to claim 1, wherein the light chain variable region of the anti-CD19 antigen binding domain comprises the amino acid sequence of SEQ ID NO: 4; and/or the heavy chain variable region of the anti-CD19 antigen binding domain comprises the amino acid sequence of SEQ ID NO: 5.

6. The chimeric antigen receptor according to claim 1, wherein the transmembrane region comprises a human CD8 transmembrane region the intracellular signaling domain comprises a human 41BB intracellular region; and/or
the hinge region comprises a human CD8 hinge region.

7. A polynucleotide molecule comprising a polynucleotide sequence encoding the chimeric antigen receptor according to claim 1.

8. A vector, comprising the polynucleotide molecule according to claim 7.

9. A lentivirus or retrovirus, comprising the polynucleotide according to claim 7.

10. A cell comprising the chimeric antigen receptor according to claim 1 or a polynucleotide molecule encoding the chimeric antigen receptor.

11. A pharmaceutical composition comprising the chimeric antigen receptor according to claim 1, a polynucleotide molecule encoding the chimeric antigen receptor or a cell expressing the chimeric antigen receptor, and a pharmaceutically acceptable excipient.

12-13. (canceled)

14. The chimeric antigen receptor according to claim 1, wherein the linker sequence comprises (GGGGS)ₘ, wherein m is 1 or 2.

15. The chimeric antigen receptor according to claim 3, wherein the anti-CD22 scFv comprises VH-(GGGGS)ₙ-VL.

16. The chimeric antigen receptor according to claim 3, wherein the anti-CD19 scFv is VH-(GGGGS)ₖ-VL, wherein n, p, q, h, k, r, s, t and v are each independently 2, 3 or 4.

17. The chimeric antigen receptor according to claim 6, wherein the CD8 transmembrane region has the amino acid sequence of SEQ ID NO: 8 or SEQ ID NO: 9.

18. The chimeric antigen receptor according to claim 6, wherein the intracellular signaling domain further comprises a human CD3ζ intracellular region.

19. The chimeric antigen receptor according to claim 6, wherein the human CD8 hinge region has the amino acid sequence of SEQ ID NO: 6 or SEQ ID NO: 7.

20. A method of treating a disease mediated by cells expressing CD19 and/or CD22 in a subject in need thereof, comprising administering to the subject the pharmaceutical composition of claim 11.

21. The method of claim 20, wherein the disease is a cancer.

22. A bispecific chimeric antigen receptor comprising, in an order from N terminus to C terminus: an anti-CD22 antigen binding domain comprising a heavy chain variable region (VH) comprising the amino acid sequence of SEQ ID NO: 2 and a light chain variable region (VL) comprising the amino acid sequence of SEQ ID NO: 3, a linker sequence comprising (GGGGS)m and m is 1 or 2, an anti-CD19 antigen binding domain comprising a VH comprising the amino acid sequence of SEQ ID NO: 5 and a VL comprising the amino acid sequence of SEQ ID NO: 4, a hinge region comprising the amino acid sequence of SEQ ID NO: 7, a transmembrane region comprising the amino acid sequence of SEQ ID NO: 9, and an intracellular signaling domain comprising the amino acid sequence of SEQ ID NO: 14.


SEQUENCE LISTING (15 sequences)

SEQ ID NO: 1 - protein, 21 aa, Artificial Sequence
  Signal Peptide
MALPVTALLLPLALLLHAARP

SEQ ID NO: 2 - protein, 116 aa, Artificial Sequence [sequence omitted]

SEQ ID NO: 3 - protein, 113 aa, Artificial Sequence
  CD22 scFv VL
DIQLTQSPSSLSASVGDRVTMSCKSSQSVLYSANHKNYLAWYQQKPGKAPKLLIYWASTRESGVPSRFSGSGSGTDFTFTISSLQPEDIATYYCHQYLSSWTFGGGTKLEIKR

SEQ ID NO: 4 - protein, 123 aa, Artificial Sequence
  CD19 scFv VL
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITRADAAPTVSIFPPSSN

SEQ ID NO: 5 - protein, 119 aa, Artificial Sequence
  CD19 scFv VH
EVKLQESGPGLVAPSQSLSVTCTVSGVSLPDYGVSWIRQPPRKGLEWLGVIWGSETTYYNSALKSRLTIIKDNSKQVFLKMNSLQTDDTAIYYCAKHYYYGGSYAMDYWGQGTSVTVSS

SEQ ID NO: 6 - protein, 45 aa, Artificial Sequence
  CD8 hinge region
TTTPAPRPPTPAPTIASQPLSLRPEACRPAAGGAVHTRGLDFACD

SEQ ID NO: 7 - protein, 55 aa, Artificial Sequence
  CD8 hinge region
FVPVFLPAKPTTTPAPRPPTPAPTIASQPLSLRPEACRPAAGGAVHTRGLDFACD

SEQ ID NO: 8 - protein, 24 aa, Artificial Sequence
  CD8 transmembrane region
IYIWAPLAGTCGVLLLSLVITLYC

SEQ ID NO: 9 - protein, 28 aa, Artificial Sequence
  CD8 transmembrane region
IYIWAPLAGTCGVLLLSLVITLYCNHRN

SEQ ID NO: 10 - protein, 751 aa, Artificial Sequence
  Bispecific CAR
MALPVTALLLPLALLLHAARPQVQLVQSGAEVKKPGSSVKVSCKASGYTFTSYWLHWVRQAPGQGLEWIGYINPRNDYTEYNQNFKDKATITADESTNTAYMELSSLRSEDTAFYFCARRDITTFYWGQGTTVTVSSGGGGSGGGGSGGGGSDIQLTQSPSSLSASVGDRVTMSCKSSQSVLYSANHKNYLAWYQQKPGKAPKLLIYWASTRESGVPSRFSGSGSGTDFTFTISSLQPEDIATYYCHQYLSSWTFGGGTKLEIKRGGGGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITRADAAPTVSIFPPSSNGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 11 - protein, 107 aa, Artificial Sequence
  anti-CD22 VL
DIQMTQTTSSLSASLGDRVTISCRASQDISNYLNWYQQKPDGTVKLLIYYTSILHSGVPSRFSGSGSGTDYSLTISNLEQEDFATYFCQQGNTLPWTFGGGTKLEIK

SEQ ID NO: 12 - protein, 123 aa, Artificial Sequence
  anti-CD22 VH
EVQLVESGGGLVKPGGSLKLSCAASGFAFSIYDMSWVRQTPEKRLEWVAYISSGGGTTYYPDTVKGRFTISRDNAKNTLYLQMSSLKSEDTAMYYCARHSGYGSSYGVLFAYWGQGTLVTVSA

SEQ ID NO: 13 - protein, 736 aa, Artificial Sequence
  Bispecific CAR
MALPVTALLLPLALLLHAARPDIQMTQTTSSLSASLGDRVTISCRASQDISNYLNWYQQKPDGTVKLLIYYTSILHSGVPSRFSGSGSGTDYSLTISNLEQEDFATYFCQQGNTLPWTFGGGTKLEIKGGGGSGGGGSGGGGSEVQLVESGGGLVKPGGSLKLSCAASGFAFSIYDMSWVRQTPEKRLEWVAYISSGGGTTYYPDTVKGRFTISRDNAKNTLYLQMSSLKSEDTAMYYCARHSGYGSSYGVLFAYWGQGTLVTVSAGGGGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 14 - protein, 42 aa, Artificial Sequence
  41BB intracellular region
KRGRKKLLYIFKQPFMRPVQTTQEEDGCSCRFPEEEEGGCEL

SEQ ID NO: 15 - protein, 112 aa, Artificial Sequence
  CD3z intracellular region
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR